SEQUENCE LISTING (3 sequences)

SEQ ID NO: 1 - DNA, 1143 bp, Homo sapiens
atgggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctaa

SEQ ID NO: 2 - DNA, 18 bp, Artificial Sequence
  chemically-synthesized primer sequence
atgggcctgggcttgtcc

SEQ ID NO: 3 - DNA, 53 bp, Artificial Sequence
  chemically-synthesized primer sequence
ggggtaccttatttttcaaactgcggatggctccaggcagccagctcagccat

(19) United States
(12) Patent Application Publication
(10) Pub. No.: US 2007/0212752 A1
(43) Pub. Date: Sep. 13, 2007

(54) Cell-free protein synthesis for controlling introduction of modification group into protein

(75) Inventors: Takashi Suzuki, Kyoto (JP); Masaaki Ito, Kyoto (JP); Toru Ezure, Kyoto (JP); Masamitsu Shikata, Kyoto (JP); Shinichiro Kobayashi, Kyoto (JP); Toshihiko Utsumi, Yamaguchi (JP)

Correspondence Address: SUGHRUE MION, PLLC, 2100 PENNSYLVANIA AVENUE, N.W., SUITE 800, WASHINGTON, DC, 20037, US

(73) Assignees: Shimadzu Corporation, Kyoto-fu (JP); National University Corporation Yamaguchi University, Yamaguchi (JP)

(21) Appl. No.: 11/700,823

(22) Filed: Feb. 1, 2007

(30) Foreign Application Priority Data
Feb. 2, 2006 (JP) 2006-026222

(51) Int. Cl. C12P 21/06; C12N 5/06; C07K 14/47

(52) U.S. Cl. 435/68.1; 435/348; 530/350

(57) ABSTRACT

It is intended to provide a method capable of introducing an arbitrary modification group into a synthetic protein and a method capable of easily controlling the syntheses of a modified protein and an unmodified protein. The present invention provides a method of cell-free protein synthesis comprising: performing cell-free protein synthesis using as a reaction solution of cell-free protein synthesis, a mixture solution of an extract derived from a eukaryotic cell and a reagent solution comprising as a substrate, a substance having a desired group to be introduced as a post-translational modification group; and thereby obtaining a protein comprising the post-translational modification group introduced therein. Preferably, the group to be introduced is an acyl group of fatty acid, and the form of the post-translational modification is lipid modification at the N-terminus of the protein.

### BACKGROUND ART

1. Technical Field

The present invention relates to a method for controlling the introduction of a modification group into a protein and a method for synthesizing a protein having a non-natural modification group by using a cell-free protein synthesis system.

2. Description of the Related Art

The post-translational modification of proteins plays an important role in the localization, stability, and functional expression of the proteins. Baculovirus expression systems or expression systems using live cells such as cultured mammalian cells (hereinafter, also referred to as “cell systems”) have currently been utilized widely for obtaining post-translationally modified proteins. Proteins expressed using cell systems undergo the same modification as post-translational modification in cells serving as hosts. Specifically, post-translational modification in the cell systems relies on hosts thereof

For example, the use of the cell systems of eukaryotes mostly produces modified proteins. The following method has been used for obtaining proteins that are not modified (hereinafter, “unmodified proteins”) by using this cell system: for example, Utsumi, T. et al ((2003). FEBS Letters, 539, 37-44) have used a method wherein the second glycine is changed into alanine in genetic-engineering manners to prevent N-myristoylation from occurring in a cell system that expresses N-myristoylated proteins.

On the other hand, the use of the cell systems of prokaryotes produces all proteins in unmodified forms. However, modified proteins cannot be obtained by using this cell system.

As described above, the cell systems of eukaryotes mostly synthesize modified proteins and, if producing unmodified proteins, involve mutations in the proteins themselves. The cell systems of prokaryotes cannot produce modified proteins. Therefore, it is impossible to control the synthesis of modified proteins and the synthesis of natural unmodified proteins.

Various forms of post-translational modification have been known which are received at the N-termini of proteins expressed using the cell systems of eukaryotes. In this case, their lipid modification is usually N-myristoylation. Specifically, when a lipid-modified protein is obtained using the cell system of a eukaryote, a modification group at the N-terminus thereof is usually only an N-myristoyl group having 14 carbon atoms. Thus, it is impossible to introduce an arbitrary modification group into the obtained protein.

### SUMMARY OF THE INVENTION

Thus, an object of the present invention is to provide a method capable of introducing an arbitrary modification group into a synthetic protein and a method capable of easily controlling the syntheses of a modified protein and an unmodified protein.

The present invention encompasses inventions described below. A “protein” used herein includes even oligopeptides and polypeptides.

[A] The following (1) to (8) relate to a method of cell-free protein synthesis:

(1) A method of cell-free protein synthesis comprising: performing cell-free protein synthesis using as a reaction solution of cell-free protein synthesis, a mixture solution of an extract derived from a eukaryotic cell and a reagent solution comprising as a substrate, a substance having a desired group to be introduced as a post-translational modification group; and thereby obtaining a protein comprising the post-translational modification group introduced therein.

(2) The method of cell-free protein synthesis according to (1), wherein the desired group to be introduced as a post-translational modification group is a group different from a natural post-translational modification group.

(3) The method of cell-free protein synthesis according to (1) or (2), wherein the desired group to be introduced as a post-translational modification group is an acyl group of fatty acid, and the form of the post-translational modification is lipid modification at the N-terminus of the protein.

(4) The method of cell-free protein synthesis according to (3), wherein the substance having the desired group to be introduced as a post-translational modification group is selected from fatty acid and CoA ester of fatty acid.

(5) The method of cell-free protein synthesis according to (3), wherein the substance having the desired group to be introduced as a post-translational modification group is selected from saturated fatty acid and CoA ester of saturated fatty acid.

(6) The method of cell-free protein synthesis according to any of (1) to (5), wherein the extract derived from a eukaryotic cell is an extract derived from an insect cell.

(7) The method of cell-free protein synthesis according to (6), wherein the extract derived from an insect cell is an extract derived from a cultured insect cell.

(8) The method of cell-free protein synthesis according to (7), wherein the cultured insect cell is a Spodoptera frugiperda 21 cell.

It is preferred that the extract derived from a eukaryotic cell should be substantially free from a low-molecular substance.

The following (9) relates to a protein obtained by cell-free protein synthesis:

(9) A modified protein obtained by a method of cell-free protein synthesis according to any of (1) to (8).

[B] The following (10) relates to a method for controlling the introduction of a post-translational modification group into a protein:

(10) A method for controlling the introduction of a post-translational modification group into a protein by selecting

the step of: performing cell-free protein synthesis using as a reaction solution of cell-free protein synthesis, a mixture solution of an extract derived from a eukaryotic cell substantially free from a low-molecular substance and a reagent solution comprising as a substrate, a substance having a desired group to be introduced as a post-translational modification group; and thereby obtaining a modified protein comprising the post-translational modification group introduced therein, or

the step of: performing cell-free protein synthesis using as a reaction solution of cell-free protein synthesis, a mixture solution of an extract derived from a eukaryotic cell substantially free from a low-molecular substance and a reagent solution not comprising as a substrate, the substance having a desired group to be introduced as a post-translational modification group; and thereby obtaining an unmodified protein.

(11) The method according to (10), wherein the desired group to be introduced as a post-translational modification group is a natural post-translational modification group or a group different from a natural post-translational modification group.

The following (12) relates to a protein obtained by cell-free protein synthesis:

(12) A modified protein and/or an unmodified protein obtained by a method according to (10) or (11).

The following (13) relates to a kit of cell-free protein synthesis:

(13) A reaction kit of cell-free protein synthesis comprising as items, both an extract derived from a eukaryotic cell substantially free from a low-molecular substance and a substance having a group to be introduced as a post-translational modification group.

The present invention can provide a method capable of introducing an arbitrary modification group into a synthetic protein and a method capable of easily controlling the syntheses of a modified protein and an unmodified protein.

### BRIEF DESCRIPTION OF THE DRAWINGS

FIG. 1 shows a result (a) of PMF analysis of digests of proteins obtained by a reaction system a supplemented with N-myristoyl CoA as a substrate having a post-translational modification group and a result (b) of PMF analysis of digests of proteins obtained by a reaction system b not supplemented with a substrate having a post-translational modification group in Experimental Example 1;

FIG. 2 shows a result of MS/MS analysis of digests of proteins obtained by a reaction system a supplemented with N-myristoyl CoA as a substrate having a post-translational modification group in Experimental Example 1; and

FIG. 3 shows results (c), (d), and (e) of PMF analysis of digests of proteins obtained by: a reaction system c supplemented with lauroyl CoA as a substrate having a post-translational modification group; a reaction system d supplemented with decanoyl CoA as the substrate; and a reaction system e supplemented with octanoyl CoA as the substrate, respectively, in Example 1, along with the results (a) and (b) shown in FIG. 1.

### DETAILED DESCRIPTION OF THE PREFERRED EMBODIMENTS

The present invention relates to [A] a method of cell-free protein synthesis for obtaining a protein comprising a desired post-translational modification group introduced therein and [B] a method for controlling the introduction of a post-translational modification group into a protein.

[A] Method for obtaining protein comprising desired post-translational modification group introduced therein

The present invention provides a method for obtaining a protein comprising a desired post-translational modification group introduced therein. In this method, an arbitrary modification group is introduced into a protein by using a reaction solution for cell-free protein synthesis comprising as one of substrate components in a cell-free protein synthesis system, a substance having a desired group to be introduced as a post-translational modification group into an obtained synthetic protein.

This method is particularly usefully used when the desired group to be introduced as a post-translational modification group is a group different from a natural post-translational modification group (i.e., a non-natural post-translational modification group). In this context, a currently known natural post-translational modification group is only a myristoyl group that binds to a glycine residue, in terms of an N-terminal lipid modification group. Other modification groups include an acetyl group that binds to an N-terminal amino acid residue, a palmitoyl group that occurs at an intramolecular cysteine residue, and farnesyl and geranylgeranyl groups that occur at a C-terminal cysteine residue.

Previously, it was impossible to introduce post-translational modification groups other than groups known as natural post-translational modification groups into proteins. However, the present invention makes it possible to introduce as non-natural post-translational modification groups, even post-translational modification groups other than those described above. This makes it possible to obtain a modified protein whose post-translational modification group is a non-natural type.

When the non-natural post-translational modification group is introduced, the present invention is useful for N-terminal protein lipidation as the form of the post-translational modification of interest. In this case, the post-translational modification group includes acyl groups of fatty acid except for a myristoyl group (saturated fatty acid having 14 carbon atoms). The acyl groups of fatty acid specifically include acyl groups of fatty acid having 6 to 16 carbon atoms, preferably 8 to 14 carbon atoms. The fatty acid may be unsaturated fatty acid or saturated fatty acid. The unsaturated fatty acid includes unsaturated fatty acid having at least one double bond or triple bond. The acyl group of fatty acid may be an acyl group of fatty acid having carbon substituted by other substances. In this case, the substances to be substituted specifically include oxygen, sulfur, nitrogen, and benzene.

To introduce such a post-translational modification group, a substance having the post-translational modification group is used as a substrate. Such a substance can be selected from, for example, fatty acid and CoA ester of fatty acid. Specifically, the substance can be selected from fatty acid having the acyl group and CoA ester of fatty acid having the acyl group.

In the present invention, a modification group to be introduced into a synthetic protein can be controlled by incorporating such a substance as a substrate into a reaction system of cell-free protein synthesis.

The usage of the substance having the post-translational modification group to be introduced is not particularly limited and can be 1 equivalent or more, for example, 1 to 20 equivalents, preferably approximately 10 equivalents, with respect to the expected amount of a protein synthesized, as a guideline. For example, when the reaction solution of cell-free protein synthesis comprises an extract of an inset cell Sf21, the expected amount of a protein synthesized is approximately 30 to 50 μg per ml of the reaction solution, as described later.

In the present invention, all components except for the substance having the post-translational modification group in the reaction solution of cell-free protein synthesis are not particularly limited as long as the components have protein synthesis activities. Examples thereof include the following substances:

An extract derived from a live cell, which is contained in the reaction solution of cell-free protein synthesis, is not particularly limited as long as the extract is derived from eukaryotes. For example, extracts derived from plants of the family Poaceae such as wheat, barley, rice, and corn, from germs of seeds of plants such as spinach, and from rabbit reticulocytes conventionally known in the art can be used without particular limitations. They can be commercially available products or can be prepared according to methods known per se. Commercially available cell extracts for protein synthesis include: rabbit reticulocyte lysate systems (manufactured by Promega) derived from rabbit reticulocytes; and wheat germ extract (manufactured by Promega) and PROTEIOS (manufactured by Toyobo Co., Ltd.) derived from wheat germs.

The reaction solution for cell-free-system protein synthesis may contain the extracts as described above and may contain extracts derived from animals proposed by the present inventors. Examples thereof include extracts derived from insect cells.

The insect cells are not particularly limited. For example, cells derived from insects such as Lepidoptera, Orthoptera, Diptera, Hymenoptera, Coleoptera, Coleoptera, Neuroptera, and Hemiptera can be used. Among them, cells derived from insects such as Lepidoptera and Hemiptera are preferable because of their many established cultured cell lines. In the present invention, the insect cells may be cells derived from any tissue. For example, hemocytes, gonad-derived cells, fat body-derived cells, embryo-derived cells, and hatched larva-derived cells can be used without particular limitations. Among them, the gonad-derived cells are preferably used which are considered to have high ability to produce proteins. Preferably, the insect cells are cultured cells. High Five (manufactured by Invitrogen) which is a cell derived from Trichoplusia ni egg cells and Sf21 (manufactured by Invitrogen) which is a cell derived from Spodoptera frugiperda ovarian cells are preferably used as the insect cells because of their high abilities to synthesize proteins in cell systems and capabilities of being cultured in serum-free media.

In the present invention, the insect cells are not limited to insect cells derived from a single tissue of a single species of insects and may be derived from several types of tissues of a single species of insects or derived from single tissues of several species of insects. Of course, the insect cells may be derived from several types of tissues of several species of insects.

A method for preparing insect cell extracts is not particularly limited. For example, a method described in Japanese Patent Laid-Open No. 2004-215651 can be used wherein insect cells suspended in a solution for extraction are rapidly frozen and then homogenized to perform extraction. This method performs cell homogenization in a mild state and therefore, is preferably used for such reasons that: the method can take components essential for cell-free protein synthesis out of cells without destroying them; the method can prevent contamination with RNase and so on from apparatuses used and so on; and the method does not bring translation reaction inhibitors of concern in cell homogenization using reagents such as surfactants.

In the present invention, extracts derived from several species of eukaryotes can also be combined for use as the extract derived from a live cell.

In the present invention, it is preferred that the extract derived from a live cell should be substantially free from a low-molecular substance.

In this context, the “low-molecular substance” does not include water and includes general substances that are removed by molecular size-based separation and purification procedures usually performed during the process of preparation of live cell extracts. Such separation and purification procedures include gel filtration, dialysis, and ultrafiltration. The low-molecular substance preferably includes substances having a molecular weight of 1000 or lower, more preferably 5000 or lower, among substances contained in the live cell extract. Such a low-molecular substance particularly includes salts. It also includes substrates having a structure likely to be introduced as an undesired group into a synthetic protein during cell-free protein synthesis.

The phrase “substantially free from a low-molecular substance” means that the low-molecular substance is removed to the extent that is achieved by the molecular size-based separation and purification procedures performed during the process of preparation of live cell extracts. Preferably, 90% or more, more preferably 98% or more of the low-molecular substances are removed.

Such removal of the low-molecular substance has the following advantages: for example, translation reaction can be performed more efficiently by removing the salts as low-molecular substances; and translation reaction can be performed without the possible unsuccessful synthesis of a post-translationally modified protein of interest or without the possible coexistence of the post-translationally modified protein of interest with a modified protein comprising an undesired group introduced therein, by removing the substrates having a structure likely to be introduced as an undesired group into a synthetic protein during cell-free protein synthesis.

Gel filtration performed as the separation and purification procedures for removing a low-molecular substance can be performed as follows: for example, in the preparation of an extract derived from a live cell, a supernatant obtained by cell homogenization and subsequent centrifugation is gel-filtrated. A fraction having absorbance of 10 or more at 280 nm (fraction with large absorption) is collected from the filtrate after the gel filtration and can be used as the extract. The gel filtration can preferably use, for example, a desalting column PD-10 (manufactured by Amersham Biosciences) and may be performed under conditions in which the column is equilibrated with a buffer solution for gel filtration according to a standard method, and a sample is then supplied to the column and eluted with the buffer solution for gel filtration. Those with appropriate composition conventionally known in the art can be used as the buffer solution for gel filtration without particular limitations. For example, a buffer solution for gel filtration containing 10 mM to 100 mM HEPES-KOH (pH 6.5 to 8.5), 50 mM to 300 mM potassium acetate, 0.5 mM to 5 mM magnesium acetate, 0.5 mM to 5 mM DTT, and 0.01 mM to 5 mM PMSF can be used. The filtrate obtained after gel filtration may be fractionated into fractions of 0.1 mL to 1 mL/fraction, as performed in usual gel filtration. Preferably, one fraction is 0.4 mL to 0.6 mL from the viewpoint of efficiently collecting a fraction having high ability to synthesize proteins. Subsequently, for example, a fraction having absorbance of 30 or more at 280 nm (fraction with large absorption) is collected from the filtrate after the gel filtration by use of an apparatus such as Ultrospec 3300 pro (manufactured by Amersham Biosciences) and can be used as the extract.

In the present invention, the cell extract is prepared in the form of an aqueous solution having a protein concentration of 1 mg/mL to 200 mg/mL, preferably 10 mg/mL to 100 mg/mL and containing 10 mM to 500 mM, preferably 50 mM to 300 mM potassium acetate, 0.1 mM to 10 mM, preferably 0.5 mM to 5 mM magnesium acetate, 0.1 mM to 10 mM, preferably 0.5 mM to 5 mM DTT, 1 μM to 50 mM, preferably 0.01 mM to 5 mM PMSF, and 5 mM to 200 mM, preferably 10 mM to 100 mM HEPES-KOH (pH 4 to 10, preferably 6.5 to 8.5). Preferably, the aqueous solution may be treated with nuclease for use.

In the method of cell-free protein synthesis of the present invention, a reaction solution prepared by adding additives necessary for cell-free-system protein synthesis to the cell extract is usually used. The additives are not particularly limited as long as the additives have conventionally been used in general in the field of cell-free-system protein synthesis.

Preferably, the reaction solution is prepared so that it contains 10(v/v)% to 80(v/v)%, particularly preferably 30(v/v)% to 60(v/v)% of the cell extract. Specifically, the extract derived from a cell is prepared at a content of preferably 0.1 mg/mL to 160 mg/mL, more preferably 3 mg/mL to 60 mg/niL, in terms of a protein concentration in the whole reaction solution. This is because a content of the extract less than 0.1 mg/mL or exceeding 160 mg/mL in terms of a protein concentration might reduce the synthesis rate of a protein of interest.

The reaction solution usually used comprises at least a potassium salt, a magnesium salt, DTT, adenosine triphosphate, guanosine triphosphate, creatine phosphate, creatine kinase, amino acid components, an RNase inhibitor, tRNA, foreign RNA, and a buffer as components except for the cell extract. This can achieve a reaction solution for cell-free-system protein synthesis further having the advantage of being capable of synthesizing proteins in large amounts in a short time.

The potassium salt in the reaction solution is not particularly limited as long as the potassium salt does not inhibit the effects of the present invention. For example, general forms such as potassium acetate, potassium carbonate, potassium hydrogen carbonate, potassium chloride, dipotassium hydrogen phosphate, dipotassium hydrogen citrate, potassium sulfate, potassium dihydrogen phosphate, potassium iodide, and potassium phthalate can be used. Among them, potassium acetate is preferably used. The potassium salt acts as a cofactor in protein synthesis reaction.

The content of the potassium salt in the reaction solution is not particularly limited. For example, a monovalent potassium salt such as potassium acetate is contained at preferably 10 mM to 500 mM, more preferably 50 mM to 150 mM, in the reaction solution from the viewpoint of storage stability. This is because a content of the potassium salt less than 10 mM or exceeding 500 mM tends to destabilize components essential for protein synthesis.

The magnesium salt is not particularly limited as long as the magnesium salt does not inhibit the effects of the present invention. For example, general forms such as magnesium acetate, magnesium sulfate, magnesium chloride, magnesium citrate, magnesium hydrogen phosphate, magnesium iodide, magnesium lactate, magnesium nitrate, and magnesium oxalate can be used. Among them, magnesium acetate is preferably used. The magnesium salt also acts as a cofactor in protein synthesis reaction.

The content of the magnesium salt in the reaction solution is not particularly limited. For example, a divalent salt such as magnesium acetate is contained at preferably 0.1 mM to 10 mM, more preferably 0.5 mM to 3 mM, in the reaction solution from the viewpoint of storage stability. This is because a content of the magnesium salt less than 0.1 mM or exceeding 10 mM tends to destabilize components essential for protein synthesis.

The dithiothreitol (hereinafter, also referred to as “DTT”) is formulated for the purpose of preventing oxidation and is contained at preferably 0.1 mM to 10 mM, more preferably 0.2 mM to 5 mM, in the reaction solution. This is because a content of the DTT less than 0.1 mM or exceeding 10 mM tends to destabilize components essential for protein synthesis.

The adenosine triphosphate (hereinafter, also referred to as “ATP”) in the reaction solution is contained at preferably 0.01 mM to 10 mM, more preferably 0.1 mM to 5 mM, in the reaction solution from the viewpoint of a protein synthesis rate. This is because a content of the ATP less than 0.01 mM or exceeding 10 mM tends to reduce a protein synthesis rate.

The guanosine triphosphate (hereinafter, also referred to as “GTP”) in the reaction solution is contained at preferably 0.01 mM to 10 mM, more preferably 0.05 mM to 5 mM, in the reaction solution from the viewpoint of a protein synthesis rate. This is because a content of the GTP less than 0.01 mM or exceeding 10 mM tends to reduce a protein synthesis rate.

The creatine phosphate in the reaction solution is a component for continuously synthesizing proteins and is formulated for the purpose of regenerating ATP and GTP. The creatine phosphate is contained at preferably 1 mM to 200 mM, more preferably 10 mM to 100 mM, in the reaction solution from the viewpoint of a protein synthesis rate. This is because a content of the creatine phosphate less than 1 mM is unlikely to regenerate sufficient amounts of ATP and GTP and consequently tends to reduce a protein synthesis rate, whereas a content of the creatine phosphate exceeding 200 mM tends to reduce a protein synthesis rate by the action of the creatine phosphate as an inhibitor.

The creatine kinase in the reaction solution is a component for continuously synthesizing proteins and is formulated for the purpose of regenerating ATP and GTP in concert with creatine phosphate. The creatine kinase is contained at preferably 1 μg/mL to 1000 μg/mL, more preferably 10 μg/mL to 500 μg/mL, in the reaction solution from the viewpoint of a protein synthesis rate. This is because a content of the creatine kinase less than 1 μg/mL is unlikely to regenerate sufficient amounts of ATP and GTP and consequently tends to reduce a protein synthesis rate, whereas a content of the creatine kinase exceeding 1000 μg/mL tends to reduce a protein synthesis rate by the action of the creatine kinase as an inhibitor.

The amino acid components contained in the reaction solution are at least 20 amino acids, that is, valine, methionine, glutamic acid, alanine, leucine, phenylalanine, glycine, proline, isoleucine, tryptophan, asparagine, serine, threonine, histidine, aspartic acid, tyrosine, lysine, glutamine, cystine, and arginine. These amino acids also include amino acids labeled with a radioisotope. Furthermore, modified amino acids may be contained in the reaction solution, if necessary. The amino acid components usually comprise approximately equal amounts of the amino acids of these types.

In the present invention, the amino acid components are contained at preferably 1 μM to 1000 μM, more preferably 10 μM to 200 μM, in the reaction solution from the viewpoint of a protein synthesis rate. This is because a content of the amino acid components less than 1 μM or exceeding 1000 μM tends to reduce a protein synthesis rate.

The RNase inhibitor in the reaction solution is formulated for the purpose of preventing protein synthesis from being inhibited due to the undesired digestion of mRNA or tRNA by cell-derived RNase coexisting in the extract during the cell-free-system protein synthesis of the present invention. The RNase is contained at preferably 0.1 U/μL to 100 U/μL, more preferably 1 U/μL to 10 U/μL, in the reaction solution. This is because a content of the RNase inhibitor less than 0.1 U/μL tends to insufficiently suppress the decomposition activity of RNase, whereas a content of the RNase inhibitor exceeding 100 U/μL tends to inhibit protein synthesis reaction.

Proteins (including peptides) encoded by the foreign mRNA in the reaction solution are not particularly limited as long as the foreign mRNA is not derived from the cells. The foreign mRNA may encode proteins having toxicity or encode glycoproteins. Whether mRNA contained in the reaction solution is foreign mRNA or is derived from the cells can be determined by: initially isolating and purifying the mRNA from an extract; then synthesizing cDNA therefrom with reverse transcriptase; analyzing the nucleotide sequence of the obtained cDNA; and comparing this nucleotide sequence with the nucleotide sequences of known foreign mRNAs.

The base number of the foreign mRNA used is not particularly limited. All the foreign mRNAs are not necessarily required to have identical base numbers as long as a protein of interest can be synthesized. The foreign mRNAs may respectively have the deletion, substitution, insertion, or addition of several bases as long as they have homologous sequences to the extent that can synthesize a protein of interest.

The foreign mRNA used in the present invention may be a commercially available product or mRNA obtained by inserting the ORF (Open reading frame) of a protein of interest into the downward region of the 5′-β globin leader sequence of a commercially available vector, for example, PT_{N}T Vector (manufactured by Promega) and performing transcription reaction with this vector. Alternatively, foreign mRNA having a cap structure added by adding a methylated ribonucleotide or the like during the transcription reaction may be used.

The foreign mRNA (hereinafter, also referred to as “mRNA”) is contained at preferably 5 μg/mL to 2000 μg/mL, more preferably 20 μg/mL to 1000 μg/mL, in the reaction solution from the viewpoint of a protein synthesis rate. This is because a content of the mRNA less than 5 μg/mL or exceeding 2000 μg/mL tends to reduce a protein synthesis rate.

The tRNA in the reaction solution comprises approximately equal amounts of tRNAs of types corresponding to the 20 amino acids described above. In the present invention, the tRNA is contained at preferably 1 μg/mL to 1000 μg/mL, more preferably 10 μg/mL to 500 μg/mL, in the reaction solution from the viewpoint of a protein synthesis rate. This is because a content of the tRNA less than 1 μg/mL or exceeding 1000 μg/mL tends to reduce a protein synthesis rate.

The buffer is formulated for the purpose of imparting buffer capacity to the reaction solution and, for example, preventing the denaturation of the extract and/or reaction products attributed to rapid changes in the pH of the reaction solution caused by the addition of an acidic or basic substance. Such a buffer is not particularly limited. For example, HEPES-KOH, Tris-HCl, acetic acid-sodium acetate, citric acid-sodium citrate, phosphoric acid, boric acid, MES, and PIPES can be used.

Preferably, the buffer used keeps the pH of the reaction solution at 4 to 10, more preferably 6.5 to 8.5. This is because pH of the reaction solution less than 4 or exceeding 10 may denature components essential for the reaction of the present invention. Among the buffers described above, HEPES-KOH (pH 6.5 to 8.5) is particularly preferably used from such a viewpoint.

The content of the buffer in the reaction solution is not particularly limited and is preferably 5 mM to 200 mM, more preferably 10 mM to 50 mM, from the viewpoint of maintaining preferable buffer capacity. This is because a content of the buffer less than 5 mM tends to cause rapid changes in pH by the addition of an acidic or basic substance and thereby denature the extract and/or reaction products, whereas a content of the buffer exceeding 200 mM tends to increase a salt concentration too much and thereby destabilize components essential for protein synthesis.

It is preferred that the reaction solution should contain EGTA. This is because EGTA contained in the reaction solution can inhibit the decomposition of components essential for the protein synthesis of the present invention by forming chelates with metal ions in the extract and thereby inactivating ribonuclease, protease, and so on. Even when the extract is treated with nuclease as described above, EGTA contained in the reaction solution can accurately prevent the nuclease from adversely affecting cell-free-system protein synthesis. The EGTA is contained at preferably 0.01 mM to 50 mM, more preferably 0.1 mM to 10 mM, in the reaction solution from the viewpoint of being capable of preferably exerting the ability to inhibit decomposition. This is because a content of the EGTA less than 0.01 mM tends to insufficiently suppress the activities of decomposing essential components, whereas a content of the EGTA exceeding 50 mM tends to inhibit protein synthesis reaction.

Specifically, it is preferred that the reaction solution used in the method of the cell-free-system protein synthesis of the present invention should be achieved in the form of an aqueous solution comprising 30(v/v)% to 60(v/v)% of the extract and comprising 50 mM to 150 mM potassium acetate, 0.5 mM to 3 mM magnesium acetate, 0.2 mM to 5 mM DTT, 0.1 mM to 5 mM ATP, 0.05 mM to 5 mM GTP, 10 mM to 100 mM creatine phosphate, 10 μg/mL to 500 μg/mL creatine kinase, 10 μM to 200 μM amino acid components, 1 U/μL to 10 U/μL RNase inhibitor, 10 μg/mL to 500 μg/mL tRNA, 20 μg/mL to 1000 μg/mL foreign mRNA, and 10 mM to 50 mM HEPES-KOH (pH 6.5 to 8.5). It is more preferred that the reaction solution should be achieved in the form of an aqueous solution further comprising 0.1 mM to 10 mM EGTA in addition to these components.

In the method of cell-free-system protein synthesis of the present invention, the reaction solution as described above is prepared to initiate synthesis at an appropriate reaction temperature. The reaction temperature is usually in the range of 10° C. to 40° C., preferably 15° C. to 30° C. This is because a reaction temperature less than 10° C. tends to reduce a protein synthesis rate, whereas a reaction temperature exceeding 40° C. tends to denature essential components. A reaction time is usually 1 hour to 72 hours, preferably 3 hours to 24 hours.

[B] Method for controlling introduction of post-translational modification group into protein

The present invention provides a method for controlling the introduction of a post-translational modification group into a protein. The method for controlling the introduction of a post-translational modification group into a protein of the present invention is specifically a method for controlling the synthesis of a modified protein and the synthesis of an unmodified protein by using cell-free protein synthesis. In this method, the introduction of a post-translational modification group into a protein is controlled by selecting either a step (B1) or (B2) below.

(B1) Cell-free protein synthesis is performed using as a reaction solution of cell-free protein synthesis, a mixture solution of an extract derived from a eukaryotic cell substantially free from a low-molecular substance and a reagent solution comprising as a substrate, a substance having a desired group to be introduced as a post-translational modification group.

In the method for controlling the introduction of a post-translational modification group into a protein of the present invention, the group to be introduced as a post-translational modification group into an obtained synthetic protein may be any of a natural post-translational modification group and a non-natural post-translational modification group.

A modified protein comprising the post-translational modification group introduced therein is obtained by performing the step (B1). Specifically, a modified protein whose post-translational modification group is a natural type or a modified protein whose post-translational modification group is a non-natural type can be obtained.

(B2) Cell-free protein synthesis is performed using as a reaction solution of cell-free protein synthesis, a mixture solution of an extract derived from a eukaryotic cell substantially free from a low-molecular substance and a reagent solution not comprising as a substrate, the substance having a desired group to be introduced as a post-translational modification group.

An unmodified protein is obtained by performing the step (B2). In the method for controlling the introduction of a post-translational modification group into a protein of the present invention, the obtained unmodified protein has a natural amino acid sequence. Specifically, the unmodified protein can be obtained without mutations.

In the method for controlling the introduction of a post-translational modification group into a protein [B], the extract derived from a live cell (extract derived from a eukaryotic cell) substantially free from a low-molecular substance is used at both the steps (B1) and (B2), as described above. The phrase “substantially free from a low-molecular substance” is as described in the method for obtaining a protein comprising a desired post-translational modification group introduced therein [A].

The natural post-translational modification group/non-natural post-translational modification group as the desired group to be introduced as a post-translational modification group, and the substance as a substrate having the group are also as described in the method for obtaining a protein comprising a desired post-translational modification group introduced therein [A].

Furthermore, the extract derived from a live cell (extract derived from a eukaryotic cell) and the mixture solution of the extract and a reagent solution are also as described in the method for obtaining a protein comprising a desired post-translational modification group introduced therein [A].

The present invention provides even a modified protein obtained by the method for obtaining a protein comprising a desired post-translational modification group introduced therein [A] and a modified protein and/or an unmodified protein obtained by the method for controlling the introduction of a post-translational modification group into a protein [B]. The proteins obtained by the methods [A] and [B] may be purified from the reaction solutions after the completion of reaction according to a usual method.

The present invention provides even a reaction kit of cell-free protein synthesis that can be used usefully for performing the method for obtaining a protein comprising a desired post-translational modification group introduced therein [A] and the method for controlling the introduction of a post-translational modification group into a protein [B]. The kit of protein synthesis of the present invention comprises as items, both an extract derived from a eukaryotic cell substantially free from a low-molecular substance and a substance having a group to be introduced as a post-translational modification group. In this context, the phrase “substantially free from a low-molecular substance”, “the extract derived from a eukaryotic cell”, “the group to be introduced as a post-translational modification group”, and “the substance having the group” are as already described. The kit of the present invention may further comprise an expression vector having the effect of promoting translation reaction, a reaction buffer for performing translation reaction under the optimum conditions, an RNase inhibitor for suppressing the undesired decomposition of mRNA, and so on.

Hereinafter, the present invention will be described specifically with reference to Example. However, the present invention is not intended to be limited to Example below.

In the present Experimental Example and Example, procedures were performed according to a protocol described in the following Steps 1. to 4.

Step 1. Construction of Expression Plasmid

PCR was performed using the cDNA (SEQ ID NO: 1) of truncated gelsolin (t-Gelsolin: the C-terminal fragment of gelsolin digested with caspase) as a template and a tGel-N primer (SEQ ID NO: 2) and a tGel-strep-C primer (SEQ ID NO: 3) to introduce a strep-tag into the C-terminal region of ORF of the t-gelsolin. The amplified DNA fragments were digested with KpnI (manufactured by Toyobo Co., Ltd.) and then inserted into EcoRV and KpnI restriction sites of a pTD1 vector (manufactured by Shimadzu Corp.). E. coli DH5α was transformed with this vector. The expression plasmid thus obtained was designated as pTD1-tGelsolin strep.

Step 2. In-vitro Transcription Reaction and mRNA Purification

The expression plasmid pTD1-tGelsolin strep constructed at the Step 1. was digested with HindIII (manufactured by Toyobo Co., Ltd.), followed by phenol-chloroform extraction and purification by ethanol precipitation. In-vitro transcription reaction was performed at 37° C. for 4 hours on a scale of 100 μl using the obtained vector (5 μg) as a template and RiboMax Large Scale RNA production System-T7 (manufactured by Promega) to thereby synthesize MRNA. The obtained reaction solution was applied to a Nick column (manufactured by Amersham Biosciences) and then eluted with sterilized water. Potassium acetate was added at the final concentration of 0.3 M to the eluted fraction, followed by ethanol precipitation. The synthesized mRNA was quantified by measuring absorbance at 260 nm and 280 nm.

Step 3. Translation Reaction and Synthetic Protein Purification

Translation reaction was performed at 25° C. for 5 hours on a scale of 1 mL using either of the following cell-free protein synthesis systems:

a reaction system not supplemented with a substrate having a post-translational modification group, i.e., a cell-free protein synthesis system constructed with a reaction system by adding the mRNA constructed at the Step 2. at the final concentration of 320 μg/mL to the reaction solution; and

a reaction system supplemented with a substrate having a post-translational modification group, i.e., a cell-free protein synthesis system constructed with a Transdirect™ insect cell (manufactured by Shimadzu Corp.) by adding the mRNA constructed at the Step 2. at the final concentration of 320 μg/mL and further the substrate having a post-translational modification group at the final concentration of 50 μM to the reaction solution.

The Transdirect™ insect cell (manufactured by Shimadzu Corp.) is a cell-free protein synthesis system using an extract of a cultured insect cell Sf21, from which a low-molecular substance has been removed by gel filtration.

The obtained reaction solution was applied to 0.5 ML Strep-Tactin superflow (manufactured by QIAGEN) equilibrated with 50 mM Tris-HCl and 300 mM NaCl, pH 8.0 (Buffer A). The column was washed with 2.5 mL Buffer A and then eluted with Buffer A containing 1.5 mL of 2 mM desthiobiotin. The eluate was collected and concentrated by ultrafiltration.

Step 4. Analysis of Post-Translational Modification Using Mass Spectrometer

A 1-μg aliquot of the purified protein obtained at the Step 3 was subjected to SDS-PAGE. After the completion of electrophoresis, the protein was detected with CBB. The band was excised and subjected to reductive alkylation and subsequent In-gel trypsin digestion. The digested peptide was extracted from the gel by use of 50% acetonitrile-0.1% TFA aqueous solution (v/v/v). The extracted digested peptide was exsiccated by drying under reduced pressure and then dissolved in 10 μL of 50% acetonitrile-0.1% TFA aqueous solution (v/v/v). The sample thus obtained was analyzed with AXIMA CFR-plus (manufactured by Shimadzu Corp.) or AXIMA-QIT (manufactured by Shimadzu Corp.).

Experimental Example 1. Control of introduction of modification into protein

[Protein Synthesis Using Cell-Free Protein Synthesis System Comprising Substrate Having Post-Translational Modification Group]

A reaction system a supplemented with N-myristoyl CoA (manufactured by SIGMA) as a substrate having a post-translational modification group was constructed at the Step 3. of the protocol, and the obtained sample was analyzed by peptide mass finger printing (PMF) with AXIMA CFR-plus and by MS/MS with AXIMA-QIT at the Step 4.

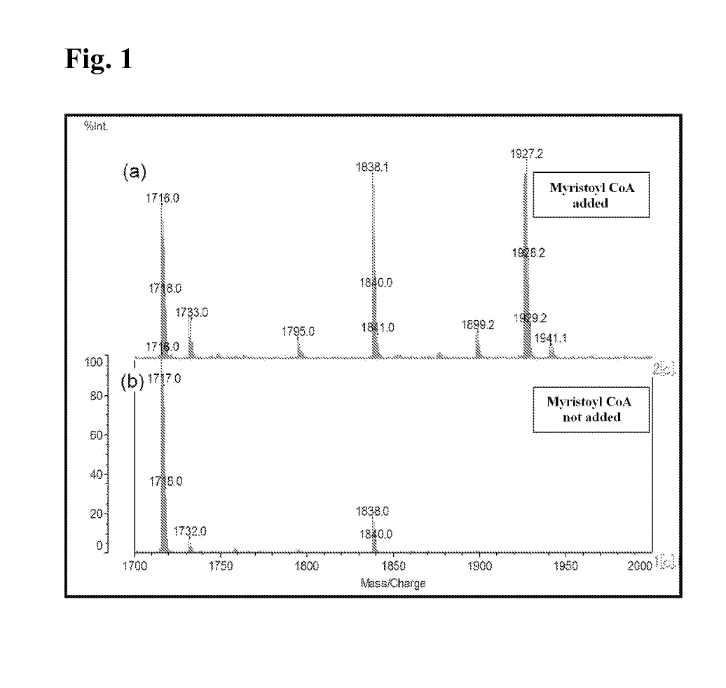

A mass spectrum obtained by PMF analysis is shown in FIG. 1(a). On the other hand, Table 1 shows the theoretical MS values of those having a mass of 1700 to 2000 among peptide fragments obtained by digesting the t-Gelsolin having the C-terminal strep-tag with trypsin.

TABLE 1

| | Theoretical | | Theoretical MS value |
|---|---|---|---|
| Position | MS value | Modification form | after modification |
| 1-17 | 1846.948 | — | — |
| 196-213 | 1837.8966 | — | — |
| 2-17 | 1715.9075 | Myristoylation | 1926.1059 |
| 374-388 | 1715.821 | — | — |

When the result of FIG. 1(a) was checked against the theoretical values of Table 1, a peak corresponding to a mass of 1926.1 was detected which was considered to be a peptide fragment generated by N-myristoylation occurring in the N-terminal peptide fragment (position Nos. 2 to 17, a mass of 1715.9) of the t-Gelsolin from which initiation methionine was eliminated. This peptide fragment indicated as a peak having a mass of 1926.1 was considered to be generated by N-myristoylation attributed to N-myristoyl CoA as a substrate.

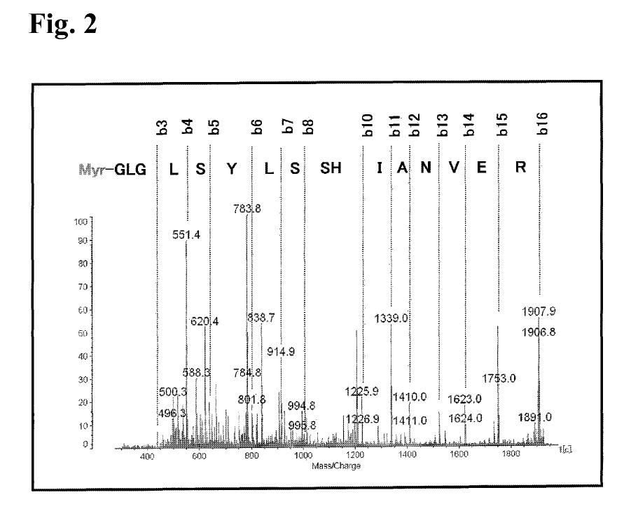

Thus, the peak having a mass of 1926.1 was subjected as a precursor ion to MS/MS analysis to thereby identify the internal amino acid sequence thereof. The result is shown in FIG. 2. The result of FIG. 2 revealed that the peak of 1926.1 obtained in FIG. 1(a) was an N-myristoylated N-terminal peptide fragment.

[Protein Synthesis Using Cell-Free Protein Synthesis System Free from Substrate Having Post-Translational Modification Group]

On the other hand, a reaction system b not supplemented with a substrate having a post-translational modification group was constructed at the Step 3 of the protocol, and the obtained sample was analyzed by PMF with AXIMA CFR-plus at the Step 4. The obtained mass spectrum is shown in FIG. 1(b). As shown in FIG. 1(b), only the N-terminal peptide fragment (position Nos. 2 to 17, a mass of 1715.9) from which initiation methionine was eliminated was detected in the reaction system b not supplemented with the substrate, while no modified peptide fragment was detected.

These results revealed that the use of the cell-free protein synthesis system can control the introduction of a modification group into a protein by the presence or absence of addition of a substrate having a post-translational modification group.

Example 1. Synthesis of N-terminally lipid-modified proteins differing in carbon chain length

Three patterns: a reaction system c supplemented with lauroyl CoA (manufactured by SIGMA) as a substrate having a post-translational modification group; a reaction system d supplemented with decanoyl CoA (manufactured by SIGMA) as a substrate having a post-translational modification group; and a reaction system e supplemented with octanoyl CoA (manufactured by SIGMA) as a substrate having a post-translational modification group were constructed at the Step 3 of the protocol, and the obtained samples were separately analyzed by PMF with AXIMA CFR-plus at the Step 4.

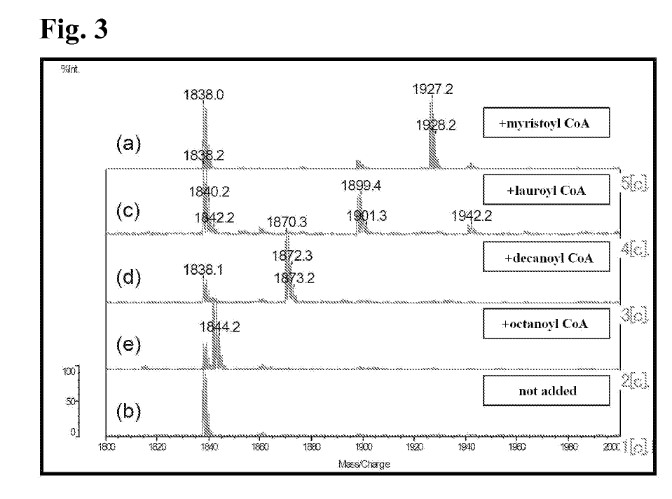

Mass spectra obtained by PMF analysis of digests of synthetic proteins obtained by the reaction systems c, d, and e are shown in FIG. 3(c), (d), and (e), respectively. FIG. 3 also shows the mass spectra (a) and (b) obtained by PMF analysis of digests of the synthetic proteins obtained by the reaction systems a and b obtained in Experimental Example 1.

As shown in FIG. 3, peptide fragments having the post-translational modification groups of the added substrates at their N-termini were separately detected. Specifically, peaks having a mass of 1842.1 (carbon chain length of 8), a mass of 1870.1 (carbon chain length of 10), and a mass of 1898.1 (carbon chain length of 12) were detected in the reaction systems supplemented with octanoyl CoA, decanoyl CoA, and lauroyl CoA, respectively.

From these results, it has been concluded that lipid modification at the N-terminus comprising an arbitrary carbon chain length depending on a substrate to be added to the reaction solution can be achieved.

What is claimed is:

1. A method of cell-free protein synthesis comprising: performing cell-free protein synthesis using as a reaction solution of cell-free protein synthesis, a mixture solution of an extract derived from a eukaryotic cell and a reagent solution comprising as a substrate, a substance having a desired group to be introduced as a post-translational modification group; and thereby obtaining a protein comprising the post-translational modification group introduced therein.

2. The method of cell-free protein synthesis according to claim 1, wherein the desired group to be introduced as a post-translational modification group is a group different from a natural post-translational modification group.

3. The method of cell-free protein synthesis according to claim 1, wherein the desired group to be introduced as a post-translational modification group is an acyl group of fatty acid, and the form of the post-translational modification is lipid modification at the N-terminus of the protein.

4. The method of cell-free protein synthesis according to claim 3, wherein the substance having the desired group to be introduced as a post-translational modification group is selected from fatty acid and CoA ester of fatty acid.

5. The method of cell-free protein synthesis according to claim 3, wherein the substance having the desired group to be introduced as a post-translational modification group is selected from saturated fatty acid and CoA ester of saturated fatty acid.

6. The method of cell-free protein synthesis according to claim 1, wherein the extract derived from a eukaryotic cell is an extract derived from an insect cell.

7. The method of cell-free protein synthesis according to claim 6, wherein the extract derived from an insect cell is an extract derived from a cultured insect cell.

8. The method of cell-free protein synthesis according to claim 7, wherein the cultured insect cell is a Spodoptera frugiperda 21 cell.

9. A modified protein obtained by a method of cell-free protein synthesis according to claim 1.

10. A method for controlling the introduction of a post-translational modification group into a protein by selecting the step of: performing cell-free protein synthesis using as a reaction solution of cell-free protein synthesis, a mixture solution of an extract derived from a eukaryotic cell substantially free from a low-molecular substance and a reagent solution comprising as a substrate, a substance having a desired group to be introduced as a post-translational modification group; and thereby obtaining a modified protein comprising the post-translational modification group introduced therein, or
the step of: performing cell-free protein synthesis using as a reaction solution of cell-free protein synthesis, a mixture solution of an extract derived from a eukaryotic cell substantially free from a low-molecular substance and a reagent solution not comprising as a substrate, the substance having a desired group to be introduced as a post-translational modification group; and thereby obtaining an unmodified protein.

11. The method according to claim 10, wherein the desired group to be introduced as a post-translational modification group is a natural post-translational modification group or a group different from a natural post-translational modification group.

12. A modified protein and/or an unmodified protein obtained by a method according to claim 10.

13. A reaction kit of cell-free protein synthesis comprising as items, both an extract derived from a eukaryotic cell substantially free from a low-molecular substance and a substance having a group to be introduced as a post-translational modification group.
